(19) United States
(12) Patent Application Publication
(10) Pub. No.: US 2011/0027376 A1
(43) Pub. Date: Feb. 3, 2011

(54) Hollow Multi-Layered Microspheres for Delivery of Hydrophilic Active Compounds

(75) Inventors: Yin Chiang Freddy Boey, Singapore (SG); Jan Ma, Singapore (SG); Zheng Wang, Singapore (SG)

Correspondence Address: Pabst Patent Group LLP, 1545 PEACHTREE STREET NE, SUITE 320, ATLANTA, GA, 30309, US

(73) Assignee: NANYANG TECHNOLOGICAL UNIVERSITY

(21) Appl. No.: 12/747,639

(22) PCT Filed: Dec. 2, 2008
(86) PCT No.: PCT/SG08/00460

§ 371 Date: Sep. 3, 2010

Related U.S. Application Data
(60) Provisional application No. 61/007,235, filed on Dec. 11, 2007.

(51) Int. Cl. A61K 9/14; A61K 33/00; A61K 39/00; A61K 38/02; A61P 25/16; A61P 25/28; A61P 25/00; A61P 3/04; A61P 9/00; A61P 3/00; A61P 3/10; A61P 35/00; A61P 37/06; A61P 9/12; A61P 27/02

(52) U.S. Cl. 424/499; 424/489; 424/600; 424/184.1; 514/1.1

(57) ABSTRACT

The present invention refers to a method of synthesizing a multi-walled microsphere comprising at least one hydrophilic active compound as well as to a multi-walled microsphere obtained by the method of the present invention. The present invention further refers pharmaceutical compositions including multi-walled microspheres of the present invention.

### CROSS-REFERENCE TO RELATED APPLICATIONS

This application claims the benefit of priority of U.S. provisional application No. 61/007,235, filed Dec. 11, 2007, the contents of each being hereby incorporated by reference in its entirety for all purposes.

### FIELD OF THE INVENTION

The present invention refers to a method of synthesizing a multi-walled microsphere comprising at least one hydrophilic active compound. The present invention also refers to a multi-walled microsphere obtained by the method of the present invention. The present invention further refers to pharmaceutical compositions including multi-walled microspheres of the present invention.

### BACKGROUND OF THE INVENTION

Controlled delivery of substances such as drugs, insecticides, fertilizers indicators and other active compounds can be achieved by a variety of processes. In one type of delivery system, a polymeric microcapsule or sphere is formed around or incorporating the active compound to be delivered.

For example, the use of devices for controlled release of drugs is a thriving area of research today. There are three major advantages of controlled release devices: (1) Most drugs are therapeutically effective only within a certain concentration window. The effort to maintain the drug concentration within Minimum Efficacious Concentration (MEC) and the Minimum Toxic Concentration (MTC) for the entire duration of the treatment is one of the major reasons to consider controlled release options. (2) Another advantage of controlled release is reduced dosage frequency and increased patient compliance. (3) By using controlled release devices, it is possible to target specific sites in the body, thereby eliminating a systemic presence of the therapeutic and avoiding the gastrointestinal tract for administration.

Usually, loaded microspheres can be fabricated with emulsion methods and spray drying. However, for drug-loaded microspheres, the high initial burst release that refers to the initial large bolus of drug released upon placing such microspheres in the release medium is one of the major challenges in developing such systems, especially for microspheres loaded with hydrophilic drugs. Too high a burst reduces the effective lifetime of the drug delivery device, reducing its effectiveness both therapeutically and economically. Even worse, excessive initial release rates can result in drug levels close to or exceeding toxic threshold levels. Although people have developed a new method to limit the burst release by using double-walled microspheres, with an inner drug loaded polymer core covered by an outer polymer layer without drugs (U.S. Pat. No. 4,861,627), the release profile of these microspheres contains typically three phases where the characteristic lag phase following the low initial burst can be over 20 days. Another drawback of such systems is that the model drug needs to be added in solid state during fabrication process, and thereby reducing the drug loading efficiency and limiting its applications for some therapeutic proteins that are originally in solution with protective excipients.

Therefore, a need exists for improved delivery systems providing an efficient and sustainable delivery of a broad spectrum of active compounds.

### SUMMARY OF THE INVENTION

In a first aspect, the present invention refers to a method of synthesizing a multi-walled microsphere comprising at least one hydrophilic active compound or substance, wherein the method includes:
providing a first solution comprising a first polymer dissolved therein in an organic solvent;
providing a second solution comprising a second polymer dissolved therein in an organic solvent, wherein the second polymer is more hydrophobic and has a higher intrinsic viscosity than the first polymer;
emulsifying at least one hydrophilic active compound dissolved in a polar solvent with said first solution to obtain a first emulsion;
emulsifying said second solution with said first emulsion to obtain a second emulsion; and
emulsifying said second emulsion with an aqueous solution comprising a stabilizer and mixing said stabilizer comprising solution to allow evaporation of said organic solvent.

In another aspect the present invention refers to a hollow microsphere encapsulating at least one hydrophilic active compound; wherein the microsphere is characterized in that it comprises a first inner polymer layer and a second outer polymer layer; and wherein the polymer of the second polymer layer has a higher hydrophobicity and a higher intrinsic viscosity than the polymer of the inner polymer layer.

In another aspect the present invention refers to a hollow microsphere manufactured by a method of the present invention and encapsulating at least one hydrophilic active compound.

In still another aspect, the present invention refers to a pharmaceutical composition comprising a hollow microsphere encapsulating at least one hydrophilic active compound obtained by a method or the present invention or a hollow microsphere of the present invention.

### BRIEF DESCRIPTION OF THE DRAWINGS

The invention will be better understood with reference to the detailed description when considered in conjunction with the non-limiting examples and the accompanying drawings, in which:

FIG. 1 shows a schematic overview of the method of the present invention indicating the separate steps of manufacturing multi-walled microspheres loaded with a hydrophilic active compound.

FIG. 2 shows a specific example for the manufacture of a double-walled microsphere of the present invention incorporating bovine serum albumin (BSA) as active test compound. This specific example is referred to in detail in the experimental section of the present invention.

FIG. 3 is a SEM picture showing the surface morphology of microspheres fabricated with PLGA 80/20 (outer layer) and PLGA 75/25 (inner layer). The microspheres obtained according to the method of the present invention have a smooth and spherical surface morphology with a mean particle diameter of between about 20 to 1000 μm.

FIG. 4 is a SEM picture showing the cross-section of an active compound loaded microsphere fabricated with PLGA 80/20 (outer layer) and PLGA 75/25 (inner layer). This picture shows the internal morphology of a microsphere and demonstrates that the inner core of substantially all microspheres manufactured with the method of the present invention is hollow or comprises a void. The lower white arrow and the cross in the center indicate the inner polymer layer or core made of the first polymer and the upper arrows in FIG. 4 indicate the outer polymer layer made of the second polymer of a double-walled microsphere of the present invention. As one can see from this picture, the inner core is largely hollow and only the outer part of the inner polymer layer or inner polymer core is solid (see the lower of the two arrows pointing towards the cross in the center of the microsphere).

FIG. 5 shows a FTIR-microscope spectra identifying the polymers in the external and the internal walls of the hollow microspheres. In this example, the external or outer wall of the microsphere is made of PLA, and the internal wall or inner wall is made of a PLGA.

FIG. 6 shows a DSC thermogram of hollow double-layered microspheres made of PLGA 80/20 (external wall) and PLGA 75/25 (internal wall). The two different T_{g} (glass transition temperature) that can be clearly seen in this DSC thermogram demonstrate the phase separation phenomenon of the double-layered structure.

FIG. 7 illustrates in a diagram the cumulative release profiles of hollow double-layered microspheres with polymer ratios of PLGA 80/20 to PLGA 75/25 of 2:1 or 1:1 (w/w), expressed as the percentage of released BSA to the amount of loaded BSA. The release profiles of these microspheres demonstrate the sustained release of BSA in a controlled manner over 3 months with a low initial burst, in the absence of the usual time lag over 20 days after the initial burst release as observed in the case of other double-walled microspheres (Lee, T. H., Wang, J., Wang, C.-H., 2002, supra).

FIG. 8 are SEM pictures showing cross-section walls for hollow double-layered microspheres made of PLGA 80/20 and PLGA 75/25 after in vitro degradation for (A) 1 day; (B) 14 days; (C) 28 days. The scale bars are indicated at the right side of each SEM picture. FIG. 8 illustrates that the protein release over the long period is controlled by the degradation rate of the inner and the outer polymers and the diffusion of hydrophilic drug molecules from the residue polymer matrix. FIG. 8 shows the increased porosity and pore size on the walls of hollow double-layered microspheres in the process of hydrolytic degradation after 1 day (A), 14 days (B) and 28 days (C). FIG. 8 (A) shows the outer layer of the microsphere before the hydrolytic degradation. At this time, the polymer walls were dense and non-porous. At day 14 after degradation (FIG. 8 (B)), pores with a diameter of less than 8 μm can be distinguished clearly on the polymer walls. The number of pores on the microsphere walls (outer layer) is largely increased after 28 days of hydrolytic degradation (FIG. 8 (C)).

### DETAILED DESCRIPTION OF THE PRESENT INVENTION

In a first aspect the present invention refers to a method of synthesizing a multi-walled microsphere comprising at least one hydrophilic active compound or hydrophilic substance, wherein the method includes:
providing a first solution comprising a first polymer dissolved in an organic solvent;
providing a second solution comprising a second polymer dissolved in an organic solvent, wherein the second polymer is more hydrophobic and has a higher intrinsic viscosity than the first polymer;
emulsifying at least one hydrophilic active compound dissolved in a polar solvent with said first solution to obtain a first emulsion;
emulsifying said second solution with said first emulsion to obtain a second emulsion; and
emulsifying said second emulsion with an aqueous solution comprising a stabilizer and mixing said stabilizer comprising solution to allow evaporation of said organic solvent.

The first polymer forms the inner polymer layer (hollow core) of the microsphere of the present invention and the second polymer forms the second layer or outer layer (depending on how many polymer layers the microsphere has) engulfing or surrounding the inner polymer layer.

Previous dissolving of the hydrophilic active compound in a polar solvent before mixing it with the first solution comprising the first polymer allows to load microspheres with hydrophilic substances, which primarily could only be added to microspheres in their solid state (Lee, T. H., Wang, J., Wang, C.-H., 2002, Journal of Controlled Release, vol. 83, pp. 437). This allows, for example, loading such microspheres with an active substance, such as a protein, in a solution comprising not only the protein but also other substances, such as substances or compounds for stabilizing the protein to be encapsulated. Such additives or additional substances can, for example, reduce or eliminate the risk of losing protein stability in the process of drying the protein-loaded microsphere or dispersing protein crystals in an organic solvent before encapsulating them in a microsphere.

Besides proteins other active compounds can also be encapsulated in the microspheres of the present invention as long as they are hydrophilic. Examples for such active compounds include other pharmaceutical substances than proteins, fertilizer, an insecticide, a chemical indicator (such as a pH indicator) or even a dye.

“Hydrophilic” (from “hydro” and “phil”, literally water-loving) means that the character of a compound is determined by its tendency to penetrate in an aqueous solution or a polar solvent, such as water, and to remain in it. For example, in an aqueous solution a hydrophilic compound can transiently bond with water (H₂O) through hydrogen bonding. Typical chemical groups found in hydrophilic active compounds are carboxylate-, sufate- and sulfonate-groups or polyether chains. Active compounds comprising hydrophilic as well as hydrophobic groups are referred to as amphiphilic (amphoterics). Due to this specific lyophilic characteristic hydrophilic compounds belong to the group of surface active substances (tensides). The antipode to hydrophilic would be hydrophobic, lipophilic or nonpolar.

As already indicated by the word “emulsion”, they are obtained by emulsification. Emulsification refers to the process of emulsifying in which two immiscible liquids are mixed together (e.g. by shaking, stirring or homogenizing) forming an emulsion. Thus, an emulsion is a disperse system of two or more immiscible liquids. One of the liquids forms a dispersant (also called continuous phase) in which the other phase (also called dispersed phase) is distributed in form of fine droplets. Emulsifying of one liquid in the other results in the formation of small droplets of one liquid dispersed (separated and distributed throughout the space) in the other liquid.

Most emulsions consist of water and oil or fat as immiscible phases. Depending on the composition and ratio of the phases two distribution options exist. In case water “W” is the continuous phase and the oil “O” is the dispersed phase, the result is an “O/W emulsion” whose basic character is determined by the aqueous phase. If oil “O” is the continuous phase and water “W” the dispersed phase, the result is a “W/O emulsion”, wherein the basic character is determined by the oil.

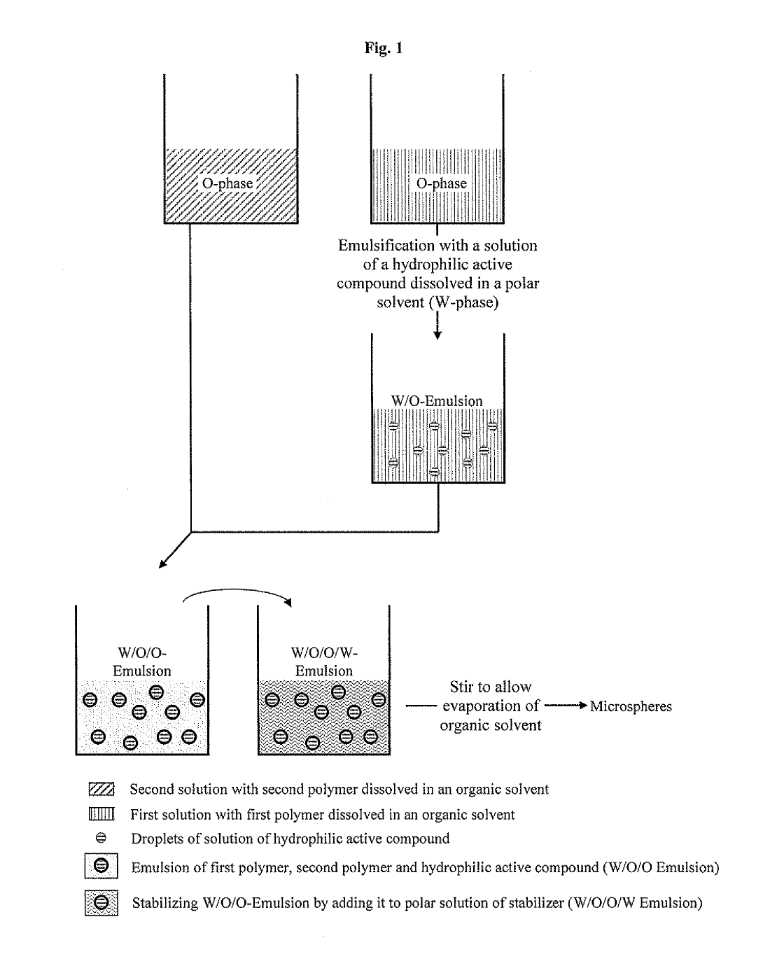

When applying this knowledge to the method of the present invention it becomes obvious that the method of the present invention uses a W/O/O/W emulsion for the manufacture of microspheres of the present invention as will be illustrated with reference to FIG. 1.

FIG. 1 is a schematic overview of the method of the present invention. At first, two solutions are provided. The first solution is an organic solvent in which at least one first polymer has been dissolved forming an O-phase. The second solution is an organic solvent in which at least one second polymer has been dissolved forming another O-phase. The difference between the first and the second solution lies in the fact that the polymer of the second solution is more hydrophobic than the polymer of the first solution.

In parallel a hydrophilic active compound is dissolved in a polar solvent and then emulsified with the first solution to obtain a first emulsion. Due to the fact that the polar solvent is dispersed in the organic solvent, i.e. the O-phase, the result is a W/O-emulsion (first emulsion). In other words the O-phase is the continuous phase (dispersant) while the hydrophilic active compound dissolved in the polar solvent forms the dispersed phase in the emulsion. Because the hydrophilic active compound is dissolved first in a polar solvent, such as an aqueous solvent, instead of adding it in solid form (such as powder or crystal) in the organic solution (O-phase) to dissolve it, the risk of destabilizing the active compound is reduced.

For example, if the active compound such as proteins are not dissolved in a polar solvent, such as an aqueous solution together with excipients first, but are mixed with an organic solution directly, as for example in a method providing a O/O/W-emulsion, proteins are highly prone to be denatured by direct contact with organic solvents. Moreover, some drugs can be obtained only in aqueous solution, such as some eye injections and eye drops. In these cases, the method of the present invention can render a chance for loading such drugs in an aqueous solution directly with a polymer, without the need of drying the drugs first, i.e. bringing it into a solid form, and thus reducing the complications in fabrication process.

In general, in case of the use of proteins as active compounds, it is essential to release them in their native conformation because the release of aggregated or denatured protein from the microsphere can result in an unwanted immune response.

Afterwards, the above described first emulsion is then emulsified with the second solution comprising the second polymer to obtain the second emulsion (W/O/O-Emulsion) (see FIG. 1). Since the second polymer of the second solution (O-phase) is more hydrophobic than the first polymer, the solubility between both polymers is different so that a phase separation between both polymers occurs in the emulsion leaving a double-walled microsphere behind in which the second polymer forms the outer wall and the first polymer encapsulating the active compound forms the inner wall.

To stabilize the microspheres formed in the second emulsion (W/O/O-Emulsion), the microspheres are emulsified in an aqueous solution comprising a stabilizer thus forming the final emulsion or stabilizer comprising solution (W/O/O/W-Emulsion) (see FIG. 1). The stabilizer positions itself at the interface between the microsphere formed and the solution comprising the stabilizer.

During solvent evaporation and subsequent washing most of this stabilizer can be removed again. Remaining small amounts of stabilizer can form part of the resultant microsphere. Therefore, in one aspect stabilizers are used which are biodegradable, such as polyvinyl alcohol (PVA) and other stabilizers mentioned further below.

This final emulsion is mixed at a suitable temperature or under vacuum to allow evaporation of the organic solvent(s). After evaporation of the solvent the microspheres can be centrifuged and washed before lyophilizing and storing them.

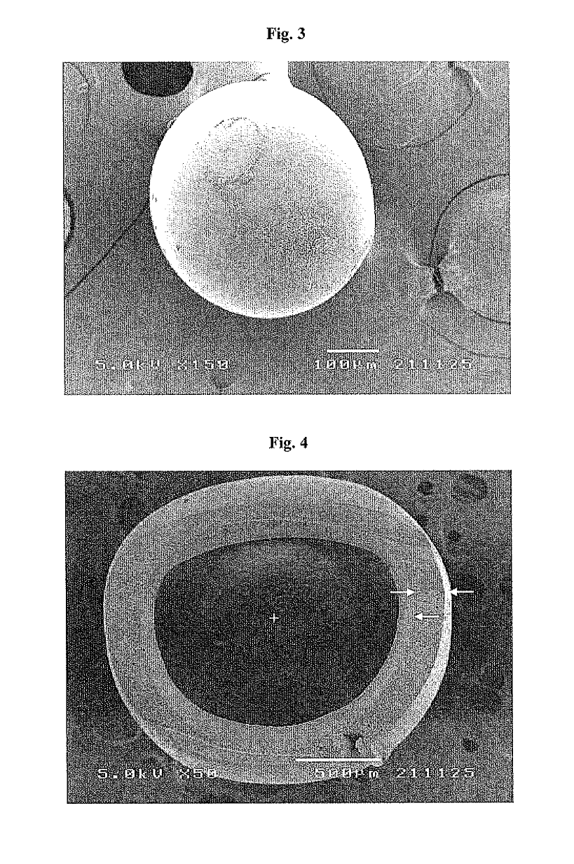

As shown in FIG. 4, substantially all microspheres obtained with the method of the present invention are hollow inside or form a void and can thus load more of the active compound than microspheres having a solid core (see e.g., Lee, T. H., Wang, J., Wang, C.-H., 2002, supra). The microspheres obtained by the method of the present invention have a smooth and spherical surface (see FIG. 3) with a mean particle diameter of between about 10 μm to about 1000 μm or 50, 100, 200, 300, 400, 500, 600 μm to about 700, 800, 900 and 1000 μm.

Upon stabilization of the active compound loaded microspheres in the solution comprising the surfactant, the final emulsion is homogenized or stirred to allow for the extraction and evaporation of the organic solvent(s) as well as hardening of the microspheres. It is also possible to subject the final emulsion to a vacuum to allow evaporation of the organic solvent(s).

Furthermore, it is possible to add a filtration and washing step for the microspheres formed and to freeze-dry them for storage purposes. In case the polymers the microspheres are made of are sensitive to moisture, they can also be stored in a desiccator to prevent hydrolytic degradation of the polymer by humidity.

For example, stirring of the final emulsion for evaporation of the organic solvent(s) can take place at a temperature between about 20 to 50° C. or 30 to 50° C. or 30 to 40° C. The time for stirring is at least about 30 or 60 min. Optionally, after stirring, the final emulsion can be stirred for another 4 or 5 to 6 or 7 hours or the final emulsion is placed under vacuum for further evaporation of the organic solvent. If the final emulsion is homogenizing, the stirring speed can be in the range of about 1000 to about 10000 rpm. If mechanical stirring is used instead of homogenizing, the stirring speed can be in the range of about 100 to about 2000 rpm.

With the microspheres of the present invention a protein loading efficiency can be as high as 85% wherein the active compound is located in the void inside the microsphere and the inner polymer layer. In general, loading rates of between about 80 to 90% or 70 to 85% are possible.

The outer polymer layer of the microspheres of the present invention has a uniform thickness (see FIG. 4), i.e. the width of the second layer formed around the first polymer layer is the same around the whole first polymer layer engulfed by the second polymer layer. Furthermore, the second polymer layer acts as a rate-limiting barrier for the release of the active compound. The inner polymer core accounts for between about 90% to 95% or 95% of the volume of the microsphere while the second or outer polymer accounts for the remaining 5 to 10%. In general it can be said that as thicker the outer polymer layer as lower the initial burst release.

As used herein the meaning of the phrases “organic solvent” and “polar solvent” is overlapping. “Organic solvent” refers to a solvent comprised of a carbon-containing chemical. “Polar solvents” refer to solvents which have generally a dielectric constant of more than 15 while solvents with a dielectric constant of less than 15 are generally considered nonpolar. However, a polar solvent can in some cases also be an organic solvent. In the context of the present invention it is referred to organic solvents in connection with the medium in which the polymers used in the method of the present invention are dissolved. The feature “polar solvent” is used to indicate the medium in which the hydrophilic active compound is dissolved. In one example, a polar solvent can comprise all polar solvents which are not organic solvents.

An example for a polar solvent that can be used to dissolve the hydrophilic active compound is water or an aqueous solution, such as a PBS buffer. The knowledge about which active compound is soluble in which polar solvent is general knowledge in the art and a person skilled in the art can therefore easily determine which polar solvent to use for a specific active compound or mixture of active compounds.

Examples for organic solvents for dissolving the polymers used in the method of the present invention are ethyl acetate (EAc), acetone, methyl ethyl ketone (MEK), tetrahydrofuran (THE), chloroform, pentane, benzene, benzyl alcohol, propylene carbonate (PC), carbon tetrachloride, methylene chloride (dichloromethane or DCM) or acetonitrile. In one example of the present invention methylene chloride (dichloromethane or DCM) is used as organic solvent.

The organic solvent or the mixture of organic solvent used for the first solvent and the second solvent is the same. Organic solvents or mixtures of organic solvents which are volatile at room temperature or which have a low boiling point are preferred, i.e. in most of the cases a boiling point equal or below 70° C. The phase separation of the polymer normally entails fast evaporation so that organic solvents with a low boiling point are preferred.

The polymers used in the method of the present invention can be biodegradable or non-biodegradable polymers. Biodegradable polymers are synthetic or natural polymers which degrade in vivo either enzymatically/non-enzymatically to produce biocompatible or non-toxic byproducts along with the progressive release of the encapsulated hydrophilic active compound.

Examples for polymers which can be used in the method of the present invention can include, but are not limited to a polyester, a polyanhydride, a polyorthoester, a polyphosphazene, a pseudopolyamino acid, a natural polymer, a polyamide, a polystyrene, ethylene vinyl acetate, polybutadiene, a polyurea, (poly) acrylate, a methacrylate, an acrylatemethacrylate copolymer, polyarylsulfone (PAS), a polyurethane, a polyalkylcyanocarylate, a polyphosphazene or copolymers and/or combinations thereof.

Further examples include, polyethylene, fluorinated polyethylene, poly-4-methylpentene, polyacrylonitrile, a polyamide-imide, polybenzoxazole, polycarbonate, polycyanoarylether, polyestercarbonate, polyether, polyetheretherketone, polyetherimide, polyetherketone, polyetherimide, polyetherketone, polyethersulfone, polyfluoroolefin, a polyimide, a polyolefin, polyoxadiazole, polyphenylene oxide, polyphenylene sulfide, polypropylene, a polysulfide, a polysulphone, polytetrafluoroethylene, a polythioether, a polytriazole, a polyvinyl, polyvinylfluoride, a silicone, urea-formaldehyde or copolymers or mixtures of these polymers.

In one example, a copolymer composed of a polyanhydride, such as Poly[bis(p-carboxy-phenoxy)propane anhydride] (CPP), polymerized with sebacic acid (SA or decanedioic acid) can also be used as polymer in the method of the present invention, wherein the ratio of CPP to SA can vary. For example, combinations, such as pCPP:SA or 20:80, 50:50 can be used. Instead of SA, it is also possible to use dodecanedioic acid (DD). Further examples for other polyanhydrides include, but are not limited to poly(malic anhydride), poly (adipic anhydride) or poly (sebacic anhydride).

Non-limiting examples for a natural polymer can include, but are not limited to a protein or a polysaccharide, wherein a protein can be, e.g., albumin, globulin, gelatin, fibrinogen, collagen or casein. Examples for suitable polysaccharides can include, but are not limited to starch, cellulose, such as cellulose ether and cellulose ester, chitosan, dextran, alginic acid, inulin or hyaluronic acid.

Non-limiting examples for polyesters can include, but are not limited to poly(∈-caprolactone) (PCL), poly(lactic acid) (PLA or PLLA), poly(glycolic acid) (PGA), polyesteramide (PEA), Poly(hydroxylbutyrate-co-hydroxyvalerate (PHS or PHBV; polyhydroxyalkanoate), an aliphatic copolyester, such as Poly(butylene succinate adipate) (PBSA), an aromatic copolyester, such as poly(butylene adipate-co-terephthalate) (PBAT), and poly(lactide-co-glycolide) acid (PLGA).

Aromatic copolyesters, such as poly(butylene adipate-co-terephthalate) (PBAT) are often based on terephthalic diacid while aliphatic copolyesters are obtained by the combination of diols such as: 1,2-ethanediol, 1,3-propanediol or 1,4-butadenediol, and dicarboxylic acid: adipic, sebacic or succinic acid. The biodegradability of aliphatic copolyester depends partly on their structure. The addition of adipic acid, which decreases the crystallinity tends to increase the biodegradation. The addition of starch filler can significantly improve the rate of degradation of an aliphatic copolyester.

For example, PLGA comprises of successive monomeric units (of glycolic or lactic acid) which are linked together in PLGA by ester linkages, thus yielding a linear, aliphatic polyester as a product. Depending on the ratio of lactide to glycolide used for the polymerization, different forms of PLGA can be obtained: these are usually identified in regard to the monomers' ratio used (e.g. PLGA 75:25 identifies a copolymer whose composition is 75% lactic acid and 25% glycolic acid. All PLGAs are amorphous rather than crystalline, PLGA degrades by hydrolysis of its ester linkages in the presence of water. The time required for degradation of a PLGA is related to the monomers' ratio used in production: the higher the content of glycolide units, the lower the time required for degradation. An exception to this rule is the copolymer with 50:50 monomers' ratio which exhibits the faster degradation (about two months). In addition, polymers that are end-capped with esters (as opposed to the free carboxylic acid) demonstrate longer degradation half-lives. Thus, the present invention also refers to PLGAs or other polymers which are chemically modificated, such as the modified PLGA mentioned above comprising end-capped esters. Furthermore, the present invention also refers to PLGAs or other polymers which have an —OH or acidic end group. Polymers modified to comprising an —OH or acidic end group degrade even faster than end-capped polymers. Polymer comprising —OH or acidic end groups are commercially available.

Polymers, such as PCL, PGA, PLA and PLGA are of interest in the biomedical area because of their biocompatibility and biodegradability properties. In particular, PLGA has been FDA approved for human therapy. When degrading, PLA and PLGA chains are cleaved in the body to monomeric acids, i.e. lactic and glycolic acids that are eliminated from the organism through Kreb's cycle as CO₂ and in urine as water.

Examples for PLGA that can be used in the method of the present invention include, but are not limited to PLGA 50:50 (lactide/glycolide molar ratio), PLGA 55:45, PLGA 85:15, PLGA 75:25, PLGA 53:47 or PLGA 80/20. However, other ratios are also possible and are also suitable to be used in the method of the present invention.

When choosing the first and second polymer to be used in the method of the present invention, it is important to ensure that a phase separation between both polymers can occur in the emulsion to form a double-walled microsphere. Phase separation occurs between incompatible polymers having a different intrinsic viscosity. On the other hand, the different hydrophobicity of the polymers ensures that the hydrophilic active compound is located in the inner polymer layer of the microsphere. Because the second polymer (forming the second or outer wall) has a higher intrinsic viscosity than the first polymer (forming the inner wall or hollow core) the polymer is forced to precipitate around the first polymer, i.e. phase separation occurs and the double-walled microsphere is formed.

According to the IUPAC definition in the Compendium of Chemical Terminology (2006) hydrophobicity is the association of non-polar groups or molecules in an aqueous environment which arises from the tendency of water to exclude non-polar molecules. In other words hydrophobicity is determined by its tendency not to penetrate in an aqueous solution. Due to this specific lyophobic characteristic, such polymers belong to the group of surface active substances (tensides). The antipode to hydrophobic (synonym: lipophilic) would be hydrophilic, lipophobic or polar. Typical hydrophobic groups are long-chain or aromatic hydrocarbon residues. In case of different PLGAs as mentioned above the first two digits indicating the polymer ratio, such as “75” for PLGA 75/25, refer to the lactide percentage. The higher ratio of lactide, the more hydrophobic the polymer. Methods for determining the hydrophobicity of a polymer are known in the art. In general, the degree of hydrophobicity of a polymer is measured by measuring the contact angle formed when a water drop is applied to the surface consisting of the polymer to be tested. As larger the contact angle (in °) of the water drop as more hydrophobic the polymer.

Polymers having a higher hydrophobicity can have also a higher molecular weight but do not need to have. A polymer having a higher hydrophobicity than another polymer does not necessarily mean that the molecular weight of the polymer with the higher hydrophobicity is also higher than for the other polymer having the lower hydrophobicity.

In one example, the molecular weight for the first polymer forming the inner core is below or equal 1.2×10⁵ g/mol and the molecular weight for the second polymer forming the second or outer wall is above or equal 1.3×10⁵ g/mol.

As mentioned, the different intrinsic viscosity of polymers used to manufacture the microsphere helps to form the multi-walled structure of the microsphere because it ensures that a phase separation takes place between the polymers having a different intrinsic viscosity. For the method of the present invention, the intrinsic viscosity of the second polymer is higher than the intrinsic viscosity of the first polymer. For example, the intrinsic viscosity for the first polymer forming the inner core is below 1.2 dl/g (120 cm³/g) and the intrinsic viscosity for the second polymer forming the second or outer wall is above 1.5 dl/g (150 cm³/g), In one example, the intrinsic viscosity of PLGA 80/20 (the second polymer) is about 1.7 to 2.6 dl/g (170 cm³/g to 260 cm³/g), while the intrinsic viscosity for PLGA 75/25 (the first polymer) is 0.93 dl/g (93 cm³/g).

The IUPAC Compendium of Chemical Terminology (2nd Edition, 1997) defines the intrinsic viscosity as follows. The intrinsic viscosity (of a polymer) is the limiting value of the reduced viscosity,

$$\frac{{η}_{i}}{c}$$

or the inherent viscosity, ηᵢₙₕ, at infinite dilution of the polymer,

$$\begin{matrix}i . e . [ η ] = {lim}_{c -> 0} \frac{{η}_{i}}{c} \\ = {lim}_{c -> 0} {η}_{inh} ( \begin{matrix}c is the concentration and \\ [ η ] is the intrinsic viscosity\end{matrix} ) .\end{matrix}$$

The term intrinsic viscosity is also known in the literature as the Staudinger index and is normally given in cm³/g. This quantity is neither a viscosity nor a pure number. The term is to be looked on as a traditional name. Any replacement by consistent terminology would produce unnecessary confusion in the polymer literature. Intrinsic viscosity is measured from the flow time of a solution through a simple glass capillary. The most useful kind of viscometer for determining intrinsic viscosity is the “suspended level” or Ubbelohde viscometer. The intrinsic viscosity is a common index for polymers that will in general be indicated for a raw polymer, and is usually measured at 25° C. in chloroform. Values for the intrinsic viscosity of polymers are generally provided by the polymer manufacturers. Normally, polymers with a higher molecular weight also have a higher intrinsic viscosity.

In one example, the molecular weight for the first polymer forming the inner core is below or equal 1.2×10⁵ g/mol and the molecular weight for the second polymer forming the second or outer wall is above or equal 1.3×10⁵ g/mol.

Suitable pairs of polymer can include, but are not limited to PLGA 75:25(1^{st} polymer)/PLGA 80/20 (2^{nd} polymer), PLGA 53:47/PLGA 80/20, PLGA 50:50/PLGA 80/20, PLGA 53:47/PLGA 75:25, PLGA 50:50/PLGA 75:25, PLGA 75:25/PLA, PLGA 53:47/PLA, PLGA 50:50/PLA, PLGA 75:25/PCL, PLGA 53:47/PCL, PLGA 55:45/PLGA 80:20, PLGA 80:20/PLGA 85:15, PLGA 75:25/PLGA 85:15, PLGA 50:50/PLGA 85:15, PLGA 55:45/PLGA 75:25, PLGA55:45/PLGA 75:25, PGLA 55:45/PCL, PGLA 55:45/PLA or PLGA 50:50/PCL.

The stabilizer used to stabilize the microsphere containing emulsion can include, but is not limited to a surfactant. The surfactant can include an amphoteric surfactant, an anionic surfactant, a cationic surfactant, a nonionic surfactant or mixtures thereof. Depending on the polymers used, the surfactant can influence the size of the microspheres formed.

Examples of an anionic surfactant can include, but are not limited to sodium dodecyl sulfate (SDS), sodium pentane sulfonate, dehydrocholic acid, glycolithocholic acid ethyl ester, ammonium lauryl sulfate and other alkyl sulfate salts, sodium laureth sulfate, alkyl benzene sulfonate, soaps, fatty acid salts or mixtures thereof.

Examples of a nonionic surfactant can include, but are not limited to poloxamers, alkyl poly(ethylene oxide), diethylene glycol monohexyl ether, polyvinyl alcohol (PVA), copolymers of poly(ethylene oxide) and poly(propylene oxide), hexaethylene glycol monohexadecyl ether, alkyl polyglucosides, digitonin, ethylene glycol monodecyl ether, cocamide MEA, cocamide DEA, cocamide TEA, fatty alcohols or mixtures thereof. In one example, polyvinyl alcohol (PVA) has been used to stabilize the microsphere emulsion.

Poloxamers, such as F127, are difunctional block copolymer surfactants terminating in primary hydroxyl groups. They are composed of a central hydrophobic chain of polyoxypropylene (poly(propylene oxide)) flanked by two hydrophilic chains of polyoxyethylene (poly(ethylene oxide)). Because the lengths of the polymer blocks can be customized, many different poloxamers exist having slightly different properties. For the generic term “poloxamer”, these copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). For the Pluronic tradename, coding of these copolymers starts with a letter to define it's physical form at room temperature (L=liquid, P=paste, F=flake (solid)) followed by two or three digits, the first digit(s) refer to the molecular mass of the polyoxypropylene core (determined from BASF's Pluronic grid) and the last digit×10 gives the percentage polyoxyethylene content (e.g., F127=Pluronic with a polyoxypropylene molecular mass of 4,000 g/mol and a 70% polyoxyethylene content). In one example, poloxamer 407 (P407) (=Pluronic F127) or F-188 or L-63 or mixtures thereof can be used.

Examples of a cationic surfactant can include, but are not limited to cetyl trimethylammonium bromide (CTAB), dodecylethyldimethylammonium bromide (D12EDMAB), didodecyl ammonium bromide (DMAB), cetylpyridinium chloride (CPC), polyethoxylated tallow amine (POEA), hexadecyltrimethylammonium p-toluenesulfonate, benzalkonium chloride (BAC), benzethonium chloride (BZT) and mixtures thereof.

Examples of an amphoteric surfactant can include, but are not limited to dodecyl betaine, sodium 2,3-dimercaptopropanesulfonate monohydrate, dodecyl dimethylamine oxide, cocamidopropyl betaine, 3-[N,N-dimethyl(3-palmitoylaminopropyl)ammonio]-propanesulfonate, coco ampho glycinate and mixtures thereof.

The thickness of the outer layer or shell and the diameter of the inner layer (inner hollow core) of the microsphere can be influenced by altering the polymer mass ratio (w/w) of the second polymer (for outer polymer layer) to the first polymer (for inner polymer layer) from 3:1 to 1:4. Other exemplary ratios include 1:3, and 1:0.5. However, ratios which are lying outside this range are also possible. The relation of polymer mass ratio to microsphere diameter is known in the art and is described, for example, by Astete, C. E., Sabliov, C. M. (2006, J. Biomater. Sci. Polymer Edn., vol. 17, no. 3, pp. 247).

Further parameters which can influence the size of the microspheres formed are the viscosity of the organic solutions in which the polymers are dissolved, the concentration of surfactant solution, the molecular mass of the polymers used, the homogenizer speed and agitation speed of mechanical stirrer and the drug entrapment in the hollow microsphere (Astete, C. E., Sabliov, C. M., 2006, supra). Generally, the higher stirring speed and the higher surfactant concentration, the smaller the particle size. Also, the use of homogenizer with high speed in the emulsion process including the surfactant can effectively reduce the particle size.

In a further example, it is possible to add further substances to the polar solvent which comprises the at least one hydrophilic active compound. Examples for such further substances include, but are not limited to an excipient, an adjuvant, an absorption enhancer, a plasticizer, stabilizing additive, a basic salt or mixtures of the aforementioned substances.

An excipient is a pharmaceutical additive, the inactive ingredients used to make up a medication. Excipients are classified by the functions they perform in a pharmaceutical dosage form. Principal excipient classifications (functions) are the following: binders, disintegrants, fillers (diluents), lubricants, glidants (flow enhancers), compression aids, colors, sweeteners, preservatives, suspensing/dispersing agents, film formers/coatings, flavors or printing inks.

For example, excipients can help the drug to disintegrate into particles small enough to reach the blood stream more quickly and still others protect the product's stability so it will be at maximum effectiveness at time of use. Although technically “inactive” from a therapeutic sense, pharmaceutical excipients are critical and essential components of a modern drug product. In many products, excipients make up the bulk of the total dosage form.

Examples for excipients, include but are not limited to magnesium stearate, lactose, microcrystalline cellulose, starch (corn), silicon dioxide, titanium dioxide, stearic acid, sodium starch glycolate, gelatin, talc, sucrose, calcium stearate, povidone, pregelatinized starch, hydroxy propyl methylcellulose, OPA products (coatings & inks), croscarmellose, hydroxy propyl cellulose, ethylcellulose, calcium phosphate (dibasic), crospovidone or shellac (and glaze).

Adjuvants are pharmacological or immunological agents that modify the effect of other agents (e.g., drugs, vaccines) while having few if any direct effects when given by themselves. Types of adjuvants include a pharmaceutic adjuvant, such as caffeine, an immunologic adjuvant and an agricultural spray adjuvant.

Adjuvants for use with agricultural pesticides have been categorized as extenders, wetting agents, sticking agents and fogging agents. An immunologic adjuvant includes inorganic, organic, oil-based or virosome adjuvants. Two common inorganic adjuvants include aluminum phosphate and aluminum hydroxide while squalene would be an example for an organic adjuvant.

An example of an immunologic adjuvant comprises Freund's adjuvant (complete (CFA) or incomplete (IFA)) which is an antigen solution emulsified in mineral oil, used as an immunopotentiator (booster of the immune system). Another example would be the Ribi adjuvants which are oil-in-water emulsions where antigens are mixed with small volumes of a metabolizable oil (squalene) which are then emulsified with saline containing the surfactant Tween 80. This system also contains refined mycobacterial products (cord factor, cell wall skeleton) as immunostimulants and bacterial monophosphoryl lipid A. A further example for an immunologic adjuvant is Titermax. Titermax represents a newer generation of adjuvants that are less toxic and contain no biologically derived materials. It is based upon mixtures of surfactant acting, linear, blocks or chains of nonionic copolymers polyoxypropylene (POP) and polyoxyethylene (POE). These copolymers are less toxic than many other surfactant materials and have potent adjuvant properties which favor chemotaxis, complement activation and antibody production.

As the name indicates “an absorption enhancer” enhances uptake of a hydrophilic active compound by the organism to which the microspheres of the present invention have been administered. Examples for an absorption enhancer include, but are not limited to surfactants, bile acids, enamine derivatives and sodium salicylate. These adsorption enhancers can for example be used to increase uptake of proteins by the organism. Examples for such proteins include, but are not limited to insulin, gastrin, lysozyme, heparin, thyrotropin-releasing hormone (TRH), enkephalins, calcitonin and glucagon.

“Plasticizers” are often used in the manufacture of microspheres to decrease T_{g} (glass transition temperature) which leads to increased diffusion rates of the drug out of the microsphere. Examples for such plasticizer include, but are not limited to phthalate esters, phosphate esters, citrate esters, sebacate esters, glycerol, triacetin, and acetylated monoglyceride. Examples of plasticizers of the above types include dimethyl phthalate, diethyl phthalate, dipropyl phthalate, dibutyl phthalate, dibutoxyethyl phthalate, dioctyl phthalate, tricresyl phosphates and triphenyl phosphates, triethyl sebacate, tributyl sebacate, acetyl tributyl citrates, or dibutyl sebacate.

Some polymers, such as PLGA, used for the manufacture of microspheres according to the method of the present invention require the addition of a basic salt into the polymer matrix to counter the acidic microenvironment. Such basic salts can include, for example, sodium bicarbonate, magnesium carbonate, magnesium hydroxide or an antacid magnesium hydroxide. In one example of the present invention magnesium carbonate (MgCO₃) has been used.

Some active compounds require the addition of stabilizing additives. For example, stabilizing additives are often used for proteins. Such stabilizing additives can include, but are not limited to further proteins, sugars, polyols, amino acids, and chelating agents.

These stabilizing additives can be added to stabilize the active compound during manufacture of the microsphere according to the method of the present invention or they are needed to stabilize the active compound after freezing and drying the manufactured microspheres.

For example, carbohydrates in particular have the ability to stabilize protein in the dried states. Sugars such as trehalose, sucrose, maltose and glucose are used as protein stabilizers for number of proteins like collagen, ribonuclease, ovalbumin. They increase the glass transition temperature of these proteins. Cyclodextrins have also been used as stabilizing additives in protein formulations.

Surfactants are also added as protein stabilizers. Addition of surfactant stabilizes a protein against denaturation during several stages from incorporation to release at site of delivery. Certain transition metals, such as zinc, had shown to confer stability on proteins and other active compounds. Lyophilization or spray drying also increases the storage stability of active compounds, such as proteins. Freeze drying itself exposes the active compound to destabilizing stresses, therefore suitable excipients and stabilizing additives are included in formulation for stability during freeze drying. Lyoprotectants such as dextran, glycols, glycerol and cyclodextrins have been found to minimize instability in some freeze-dried formulations (see, e.g., Sinha, V. R., Trehan, A., 2003, J. of Controlled Release, vol. 90, pp. 261).

The release of the at least one active compound from the microspheres is dependent both on diffusion through the polymer matrix and on polymer degradation. If during, the desired release time, polymer degradation is considerable, then the release rate may be unpredictable and erratic due to breakdown of microspheres. However, the release of encapsulated material from such systems is dependent on diffusivity through the polymer barrier, solubility of core in bulk phase, size of active compound and distribution of encapsulated material throughout the matrix, etc. Nature of polymer plays a major role in release process. Route of administration of injectable microspheres may also alter the duration of release.

Added control over delivery of the encapsulated active compound can also be achieved by employing pH-triggered release. Therefore, by the incorporation of pH-sensitive groups into the polymers used for manufacturing the microspheres, the microspheres can be targeted to various biological environments or to specific organs (Freiberg, S., Zhu, X. X., 2004, Int. J. of Pharmaceutics, vol. 282, pp. 1).

As to the route of administration, the microspheres obtained by the method of the present invention can be administered via any route depending on the desired application. Exemplary routs of administration include, for example, the oral, rectal, buccal, transdermal, nasal or ocular route.

As previously mentioned, hydrophilic active compounds which can be incorporated or encapsulated in the microspheres of the present invention can include, for example, drugs, a fertilizer, an insecticide, a chemical indicator, such as a pH indicator and pigments and dyes, such as an azo dye or leuko dye.

Insecticides can be selected from the group of chlorinated hydrocarbons, such as Endosulfan and Aldrin, organophosphates, such as Acephate and Malathion, carbamates, such as Aldicarb and 2-(1-Methylpropyl)phenyl methylcarbamate, phenothiazine, pyrethroids, such as Allethrin and Tralomethrin, neonicotinoids, such as Acetamiprid and Nithiazine, plant derived compounds, such as caffeine, Anabasine, Linalool and Pyrethrum.

Fertilizer are chemical compounds given to plants to promote growth and can include inorganic fertilizer, such as sodium nitrate, mined rock phosphate and limestone, or organic fertilizer, such as manure, slurry, worm castings, peat, seaweed, sewage and guano.

Hydrophilic drugs or pharmaceuticals which can be encapsulated can comprise all kinds of pharmaceuticals. The microspheres can comprise, for example, pharmaceuticals effective for the prevention or treatment of the gastrointestinal tract/metabolism; blood and blood forming organs; cardiovascular system; skin; reproductive system; endocrine system; infections and infestations; malignant disease; immune disease; muscles, bones, and joints; brain and nervous system; respiratory system or mixtures thereof. The microspheres can also comprise antidotes, contrast media, radiopharmaceuticals and dressings or mixtures thereof.

Examples, for some common pharmaceuticals/drugs include, but are not limited to atorvastatin, clopidogrel, enoxaparin, celecoxib, omeprazole, esomeprazole, fexofenadine, quetiapine, metoprolol and budesonide.

Encapsulation of vaccines, such as group B Streptococcus vaccine (GBS), tetanus toxoid (TT), Japanese encephalitis virus (JEV), diphtheria toxoid (DT), vibrio cholerae (VC), SPF 66 malaria vaccine, multivalent vaccines of Haemophilus influenza type b (Hib), pertussis toxin (PT), Rotavirus to name only a few, is also possible. Examples of proteins which can be encapsulated include bovine serum albumin (BSA), recombinant human epidermal growth factor (rhEGF), recombinant human erythropoietin (rhEPO), protein-C, ribozymes, vapreotide (somastatin analogue), orntide acetate (LHRH antagonist), growth factors, such as insulin like growth factor-1 (IGF-1), vascular endothelial growth factor (VEGF); ovalbumin, human chorionic gonadotropin (hCG), human serum albumin (HSA), recombinant human morphogenetic protein-2 (rhBMP) and calcitonin, to name only a few examples. Even though all those substances have been used previously, the controlled release of such substances using the microspheres, which show a highly reduced initial burst of the encapsulated substances, allow obtaining a well defined pharmacokinetic profile.

In another aspect, the present invention refers to a hollow microsphere encapsulating at least one hydrophilic active compound. The hollow microsphere
comprises a first inner polymer layer and a second outer polymer layer; and wherein
said polymer of said second polymer layer has a higher hydrophobicity than said polymer of said inner polymer layer.

The hydrophilic active compound is encapsulated in the hollow microsphere. In particular the hydrophilic active compound is located in the void formed by the first polymer and is also incorporated into the shell of the inner core formed by the first polymer. In contrast, the hydrophilic active compound is not incorporated in the second polymer layer or only in very small amounts compared with the inner microsphere core.

Due to this unique structure and distribution of active compound the hollow microsphere has an initial burst rate for the hydrophilic active compound of below 5% or 4%. Based on the initial burst release of the microspheres obtained according to the method of the present invention, it is estimated that the amount of active compound incorporated in the outer polymer layer is below 2 or 3%.

To further increase the degradation time of the microspheres of the present invention it is possible to increase the thickness of the second polymer layer as described above, for example by increasing the ratio of second to first polymer, or to add another polymer layer using the above described emulsion method or any other method, such as fluid bed coating, which is suitable to add another polymer layer to the existing two polymer layers.

In still another aspect, the present invention refers to a hollow microsphere obtained by a method of the present invention encapsulating a hydrophilic active compound as described above. Furthermore, the present invention refers to a pharmaceutical composition comprising a hollow microsphere encapsulating at least one hydrophilic active compound obtained by a method of the present invention.

The pharmaceutical composition can be used for the treatment or prevention of a disease or condition of a patient which can include cancers, autoimmune disorders, such as HIV, memory impairment, mental disorders, hypertension, cardiovascular diseases, eye diseases, such as glaucoma, and metabolic diseases. Also included are age related diseases or conditions, such as diabetes, altered blood pressure, such as high blood pressure, altered blood cholesterol levels, obesity, Alzheimer disease, Huntington disease, Parkinson disease and prion related diseases, such as Bovine Spongiform Encephalopathy (BSE).

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

### Fabrication Process of Hollow and/or Solid Microspheres

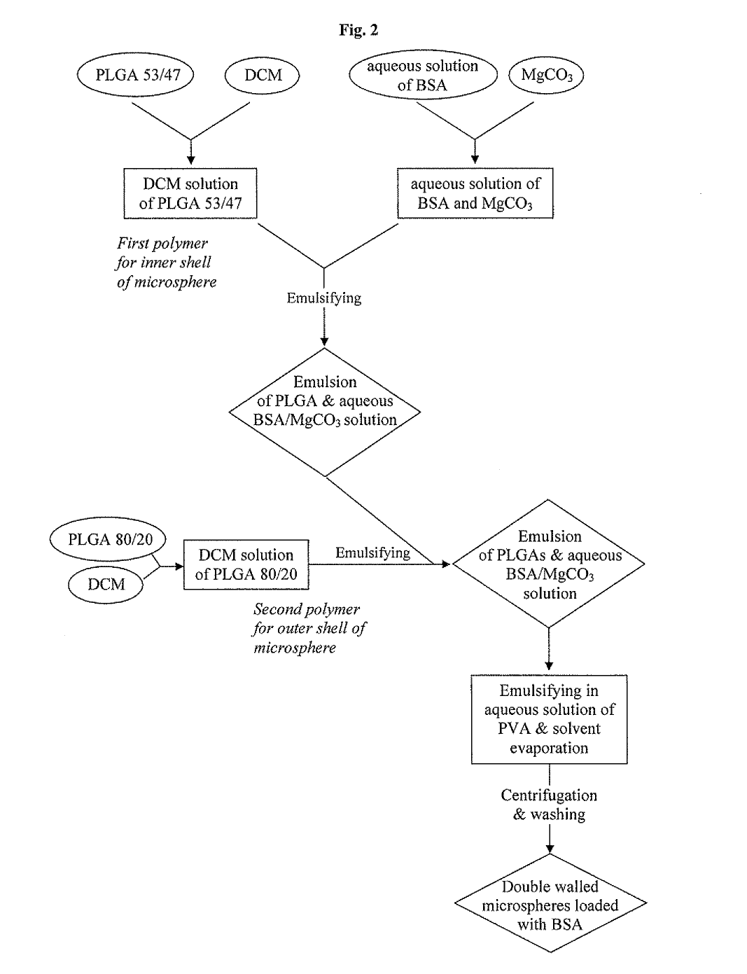

At first, 3 ml dichloromethane solution of PLGA 75/25 or PLGA 53/47, PLGA 50/50 (first solution) (166.7 mg/ml or specified otherwise later) are emulsified with 1 ml bovine serum albumin (BSA) aqueous solution (polar solvent) (50 mg/ml or specified otherwise later) and MgCO₃ powders (basic salt) (in order to neutralize the acidic degradation products of PLLA or PLGA) using a homogenizer (Ultra Turrax T8, IKA®-WERKE, Germany). The resultant emulsion is then emulsified with 3 ml (or specified otherwise) dichloromethane solution of PLGA 80/20 or PLLA (second solution) (166.7 mg/ml or specified otherwise) using the same homogenizer. This emulsion is subsequently poured into a 100 ml aqueous 0.3% PVA solution (aqueous solution with stabilizer), which is then stirred using a mechanical stirrer (BDC 1850-220, Caframo®) or a homogenizer (L4R, Silverson, USA) for 6 h at 22° C., to allow solvent evaporation. The microspheres are collected by centrifuging and washed three times with de-ionized water. The BSA loaded microspheres are lyophilized and then stored at 4° C. A schematic overview over this process is shown in FIG. 2.

Determination of Active Compound Loading of Microspheres

20 mg of the above described lyophilized microspheres are added into 2.0 ml of 0.1 M NaOH solution containing 5% (w/v) SDS and incubated for 24 h at 37° C. with occasional shaking. The mixture is then centrifuged and the supernatant is drawn to determine BSA loading using a bicinchoninic acid kit (BCA). The encapsulation efficiency is expressed as the ratio of actual-to-theoretical BSA content. The active compound loading is expressed as: % active compound loading

$$= \frac{Amount of loaded active compound}{Amount of polymer + Amount of loaded active compound} \times 100$$

In-Vitro Release of Active Compound from Microspheres

20±0.2 mg dried microspheres are placed in a centrifuge tube and dispersed in 2 ml PBS buffer (pH 7.0). The tube is placed in a 37° C. incubator for 24 h with occasional shaking. 1 ml supernatant from the tube is collected at prescribed time and analyzed for BSA content using BCA kit. Then the tube is replenished with 1 ml fresh PBS buffer (pH 7.0). The in vitro release of BSA is expressed as the percentage of protein released to the total amount of loaded protein.

Scanning Electronic Microscope (SEM)

Lyophilized microspheres are mounted onto metal stubs using double-sided adhesive tapes and then vacuum-coated with gold. The surface morphology and internal structure of microspheres are examined by SEM (Model JSM 6360A, JEOL, Japan) at 5, 10, or 15 kV.

Fourier Transformed Infra-Red (FTIR) Spectra

FTIR spectra are obtained using FTIR microscope (Bio-Rad UMA 500) connected to FTIR spectrophotometer mainframe (Bio-Rad FTS-3500 ARX) and analyzed using Bio-Rad analysis software in the mid IR range (wave number 400-4000 cm⁻¹, resolution 2 cm⁻¹). Standard microspheres of a single polymer and hollow double-layered microspheres are cross-sectioned into halves and mounted on a gold slide for examination. Ten points are randomly selected on the external and the internal walls using the software to obtain the transmission spectra.

Thermal Analysis of the Microspheres

Thermal analysis of the microspheres is performed with a differential scanning calorimeter (DSC Q10, TA instruments) equipped with a cooling system. Around 6.0 mg samples are placed in sealed aluminum pans and are subjected to a heating program from −20° C. to 100° C. for the first heating ramp, then cooled to −10° C. and reheated on the second ramp to 100° C. at a rate, of 10° C./min. Data obtained are processed on TA universal analyzer software and identified for glass transition temperature (T_{g}).

Experimental Results

Hollow double-layered microspheres loaded with an active compound as produced at the aforementioned conditions have smooth and spherical surface morphology (FIG. 3), with the mean particle diameter of between about 20 to 1000 μm. The particle size depends on the stirring speed of mechanical stirrer and the concentration of surfactant solution in the secondary emulsion process. Generally, the higher stirring speed and the higher surfactant concentration, the smaller the particle size. Also, the use of homogenizer with high speed in the secondary emulsion process can effectively reduce the particle size. The cross-section picture (FIG. 4) shows the hollow internal morphology of active compound loaded microspheres.

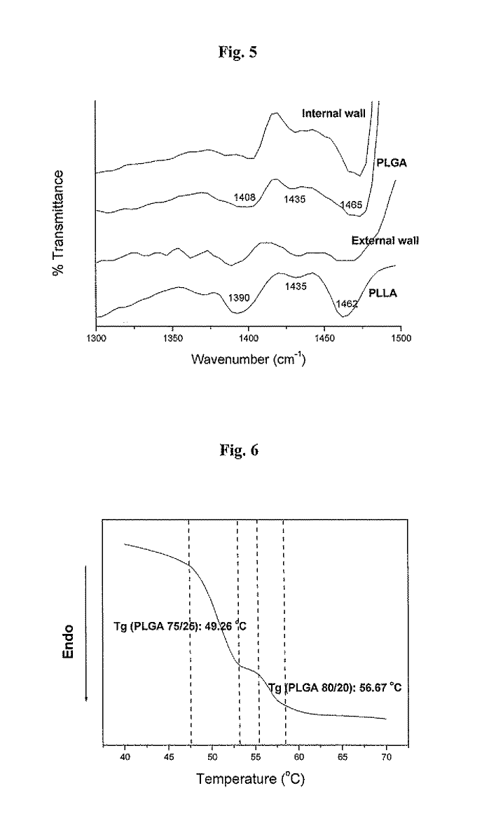

In order to identify materials of the internal and the external walls of hollow double-layered microspheres, FTIR-microscope spectra are obtained with PLA-PLGA microspheres, as the difference of FTIR spectra between PLGA 80/20 and PLGA 50/50, PLGA 53/47 or PLGA 75/25 is difficult to be clearly identified. FTIR-microscope spectra (FIG. 5) of PLLA showed a C-H bending vibration of methyl group at 1390 and 1462 cm⁻¹, while PLGA showed a C-H bending vibration of methyl group at 1408 and 1465 cm⁻¹, and an additional C-H vibration of methylene group at 1435 cm⁻¹. The spectra of the internal wall correspond to that of PLGA while the outer wall to PLA. The phase separation phenomenon of the double-layered structure can be further substantiated with the DSC thermogram of microspheres made of PLGA 80/20 and PLGA 75/25, which shows clearly two T_{g} (FIG. 6). As it is expected, the phase separation process is more difficult to be achieved with those polymer pairs that have relatively similar solubility parameters in the solvent DCM, such as PLGA 80/20 and PLGA 75/25 because of their similar physico-chemical properties, as determined in the equation (1):

$$δ = \frac{ρ \sum G}{M} \qquad ( 1 )$$

Where δ is the solubility parameter, ρ is the density of the polymers, and M is the molecular weight of the monomer unit. Group molecular attraction constants (G) have been calculated previously (Billmeyer, F. W., 1984, Textbook of polymer science, New York: John Wiley & Sons, page 25).

The finding of the complete phase separation between PLGA 80/20 and PLGA 75/25, which have similar physico-chemical parameters, predicts the phase separation between other polymer pairs that have been used in this study. In fact, similar phase separation processes have also been seen on hollow double-layered microspheres made of other polymer pairs, like for example for PLGA 53/47 and PLGA 80/20; PLGA 50/50 and PLGA 80/20.

The protein loading efficiency of these microspheres can be as high as 84.8±2.3%, with the low initial burst release of 3.3±0.6% with respect to totally loaded BSA protein. This low initial burst release has been obtained with 6 ml dichloromethane solution of PLGA 80/20 or PLLA (166.7 mg/ml) and 3 ml dichloromethane solution of PLGA 50/50, PLGA 53/47 or PLGA 75/25 (166.7 mg/ml). It is know that the initial burst release depends on the immediate diffusion of proteins from polymer matrix. As demonstrated above, these microspheres can be classified as a kind of reservoir-dispersed matrix system in which the hydrophilic active compound, such as proteins, is dispersed within the internal layer, and the outer layer acts as a rate-limiting barrier to drug release. Therefore, higher amount of PLGA 80/20 or PLLA than usual 1:1 ratio to PLGA 50/50, PLGA 53/47 or PLGA 75/25 can enhance the thickness of non-drug-loaded outer layer, and thus further depress the initial burst release. In comparison, the initial burst release of BSA from monolithic micropheres made of single polymer such as PLGA 75/25 can be over 60% (Jain, R. A., Rhodes, C. T., 2000, Eur. J. Pharm. Biopharm., vol. 50, pp. 257).

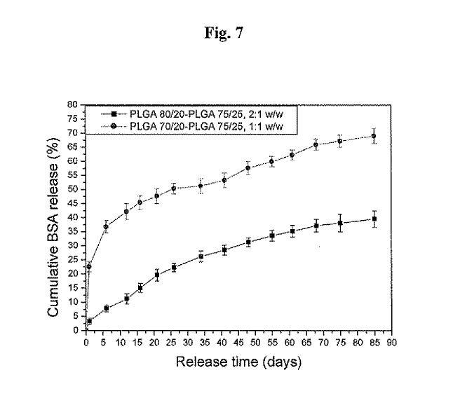

The release profiles of these microspheres (FIG. 7) demonstrate the sustained release of proteins in a controlled manner over 3 months with a low initial burst, in the absence of the usual time lag over 20 days after the initial burst release as observed in the case of other double-walled microspheres (Lee, T. H., Wang, J., Wang, C.-H., 2002, supra).

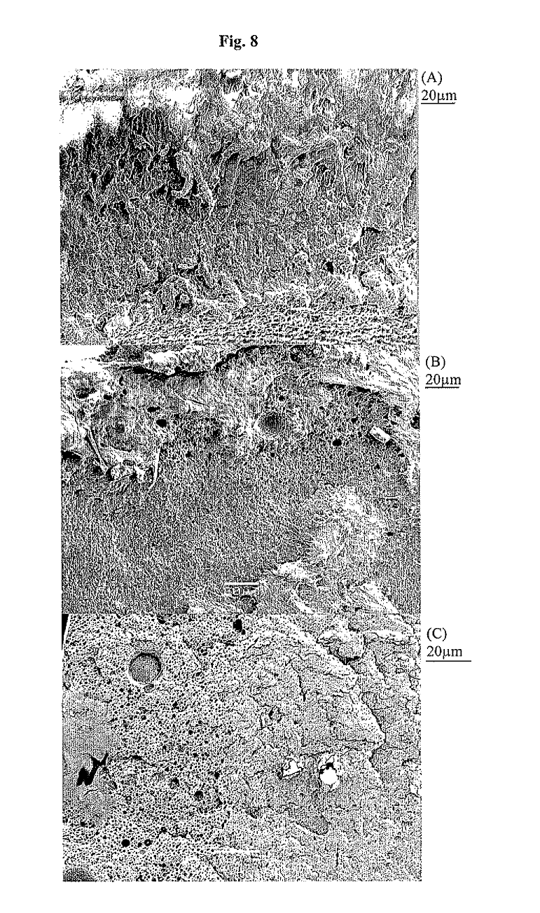

The protein release over the long period is controlled by the degradation rate of the inner and the outer polymers and the diffusion of hydrophilic drug molecules from the residue polymer matrix. FIG. 8 shows the increased porosity and pore size on the walls of hollow double-layered microspheres made of PLGA 80/20 and PLGA 75/25 in the process of hydrolytic degradation after 1 day (A), 14 days (B) and 28 days (C). FIG. 8 (A) shows the outer layer of the microsphere before the hydrolytic degradation. At this time, the polymer walls were dense and non-porous. At day 14 after degradation (FIG. 8 (B)), pores with a diameter of less than 8 μm can be distinguished clearly on the polymer walls. The number of pores on the microsphere walls (outer layer) is largely increased after 28 days of hydrolytic degradation (FIG. 8 (C)).

1. A method of synthesizing a multi-walled microsphere comprising at least one hydrophilic active compound, wherein said method comprises:
providing a first solution comprising a first polymer dissolved in an organic solvent;
providing a second solution comprising a second polymer dissolved in an organic solvent, wherein said second polymer is more hydrophobic and has a higher intrinsic viscosity than said first polymer;
emulsifying at least one hydrophilic active compound dissolved in a polar solvent with said first solution to obtain a first emulsion;
emulsifying said second solution with said first emulsion to obtain a second emulsion; and
emulsifying said second emulsion with an aqueous solution comprising a stabilizer and mixing said stabilizer comprising solution to allow evaporation of said organic solvent.

2. The method according to claim 1, wherein said polar solvent is an aqueous solution.

3. The method according to claim 1, wherein said first and said second polymer is a biodegradable polymer(s).

4. The method according to claim 1, wherein said first and said second polymer are selected from the group consisting of a polyester, a polyanhydride, a polyorthoester, a polyphosphazene, a pseudopolyamino acid, a natural polymer, a polyamide, a polystyrene, ethylene vinyl acetate, polybutadiene, a polyurea, acrylate, a methacrylate, an acrylatemethacrylate copolymer, polyarylsulfone (PAS), a polyurethane, a polyalkylcyanocarylate, a polyphosphazene, polyethylene, fluorinated polyethylene, poly-4-methylpentene, polyacrylonitrile, a polyamide-imide, polybenzoxazole, polycarbonate, polycyanoarylether, polyestercarbonate, polyether, polyetheretherketone, polyetherimide, polyetherketone, polyetherimide, polyetherketone, polyethersulfone, polyfluoroolefin, a polyimide, a polyolefin, polyoxadiazole, polyphenylene oxide, polyphenylene sulfide, polypropylene, a polysulfide, a polysulphone, polytetrafluoroethylene, a polythioether, a polytriazole, a polyvinyl, polyvinylfluoride, a silicone, urea-formaldehyde and copolymers and combinations thereof.

5. The method according to claim 4, wherein said natural polymer is selected from the group consisting of a protein and a polysaccharide.

6. The method according to claim 5, wherein said protein is selected from the group consisting of albumin, globulin, gelatin, fibrinogen, collagen and casein, and wherein said polysaccharide is selected from the group consisting of starch, cellulose, chitosan, dextran, alginic acid, inulin and hyaluronic acid.

7. (canceled)

8. The method according to claim 4, wherein said polyester is selected from the group consisting of a poly(∈-caprolactone) (PCL), poly(lactic acid) (PLA), poly(glycolic acid) (PGA), polyetherimide (PEA), Poly(hydroxylbutyrate-co-hydroxyvalerate (PHB or PHBV; polyhydroxyalkanoate), an aliphatic copolyester, an aromatic copolyester and poly(lactide-co-glycolide) acid (PLGA).

9. The method according to claim 8, wherein said PLGA is selected from a group consisting of PLGA 50:50 (lactide/glycolide molar ratio), PLGA 75:25, PLGA 53:47, PLGA 55:45, PLGA 85:15 and PLGA 80:20.

10. The method according to claim 7, wherein said first polymer and said second polymer are selected from the group of pairs of first (1^{st}) and second (2^{nd}) polymers ((1^{st})/2^{nd}) consisting of PLGA 75:25/PLGA 80/20, PLGA 53:47/PLGA 80/20, PLGA 50:50/PLGA 80/20, PLGA 53:47/PLGA 75:25, PLGA 50:50/PLGA 75:25, PLGA 75:25/PLA, PLGA 53:47/PLA, PLGA 50:50/PLA, PLGA 75:25/PCL, PLGA 53:47/PCL, PLGA 55:45/PLGA 80:20, PLGA 80:20/PLGA 85:15, PLGA 75:25/PLGA 85:15, PLGA 50:50/PLGA 85:15, PLGA 55:45/PLGA 75:25, PLGA55:45/PLGA 75:25, PGLA 55:45/PCL, PGLA 55:45/PLA and PLGA 50:50/PCL.

11. The method according to claim 9, wherein said organic solvent is dichloromethane (DCM).

12. The method according to claim 1, wherein said stabilizer is a surfactant which is selected from the group consisting of an amphoteric surfactant, an anionic surfactant, a cationic surfactant, a nonionic surfactant and mixtures thereof.

13. The method according to claim 4, wherein said polyanhydride is selected from the group consisting of poly[bis(p-carboxy-phenoxy)propane anhydride] (CPP), poly(malic anhydride), poly (adipic anhydride) and poly (sebacic anhydride).

14. The method according to claim 1, wherein said organic solvent is selected from the group consisting of ethyl acetate (EAc), acetone, methyl ethyl ketone (MEK), tetrahydrofuran (THF), chloroform, pentane, benzene, benzyl alcohol, propylene carbonate (PC), carbon tetrachloride, methylene chloride (dichloromethane or DCM) and acetonitrile.

15. The method according to claim 1, wherein the ratio of said second polymer to said first polymer is in the range between about 3:1 to 1:4.

16. The method according to claim 1, wherein said intrinsic viscosity of said second polymer is above 1.5 dl/g (150 cm³/g), or wherein said intrinsic viscosity of said first polymer is below 1.2 dl/g (120 cm³/g).

17. (canceled)

18. The method according to claim 1, wherein said second polymer has a molecular weight which is above the molecular weight of said first polymer.

19. The method according to claim 18, wherein said second polymer has a molecular weight above or equal 1.3×10⁵ g/mol, or wherein said first polymer has a molecular weight below or equal 1.2×10⁵ g/mol.

20. (canceled)

21. The method according to claim 1, wherein said polar solvent comprising the at least one hydrophilic active compound further comprises a substance selected from the group consisting of an excipient, an adjuvant, an absorption enhancer, a plasticizer, a stabilizing additive, a basic salt and mixtures of the aforementioned substances.

22. The method according to claim 1, further comprising at least one centrifugation and at least one washing step after evaporation of said organic solvent.

23. The method according to claim 1, further comprising lyophilizing said microspheres formed in said method.

24. The method according to claim 1, wherein said at least one hydrophilic active compound is selected from the group consisting of a drug, a fertilizer, an insecticide, a chemical indicator and a dye.

25. The method according to claim 24, wherein said drug is selected from the group consisting of a vaccine, a protein, an inorganic molecule and mixtures thereof.

26. The method according to claim 1, further comprising applying further polymer layers at the surface of said multi-walled microcapsule formed.

27. A hollow microsphere encapsulating at least one hydrophilic active compound; wherein said microsphere
comprises a first inner polymer layer and a second outer polymer layer; and wherein
said polymer of said second polymer layer has a higher hydrophobicity and has a higher intrinsic viscosity than said polymer of said inner polymer layer.

28. The hollow microsphere according to claim 27, wherein said hollow microsphere has a initial burst for said active compound of below 5%.

29. The hollow microsphere according to claim 27, wherein said hollow microsphere has a maximal dimension of between about 10 μm to about 1000 μm.

30. The hollow microsphere according to claim 27, wherein said hollow microsphere has a dense surface.

31. A hollow microsphere manufactured by a method according to claim 1 and encapsulating at least one hydrophilic active compound.

32. A pharmaceutical composition comprising a hollow microsphere encapsulating at least one hydrophilic active compound obtained by a method according to claim 1 or a hollow microsphere according to claim 27.

33. The pharmaceutical composition according to claim 32 for the treatment or prevention of a disease or condition of a patient selected from the group consisting of cancers, autoimmune disorders, memory impairment, mental disorders, hypertension, cardiovascular diseases, eye diseases and metabolic diseases; or for the treatment or prevention of a disease or condition selected from the group consisting of diabetes, altered blood pressure, altered blood cholesterol levels, obesity, Alzheimer disease, Huntington disease, Parkinson disease and prion related diseases.

34. (canceled)
